(19) United States
(12) Patent Application Publication
(10) Pub. No.: US 2016/0296320 A1
(43) Pub. Date: Oct. 13, 2016

(54) INTRAOCULAR LENSES UTILIZING MULTIPLE FILLING FLUIDS

(71) Applicants: Mark S. HUMAYUN, Glendale, CA (US); Charles DEBOER, Sierra Madre, CA (US); Yu-Chong TAI, Pasadena, CA (US)

(72) Inventors: Mark S. HUMAYUN, Glendale, CA (US); Charles DEBOER, Sierra Madre, CA (US); Yu-Chong TAI, Pasadena, CA (US)

(21) Appl. No.: 15/093,074

(22) Filed: Apr. 7, 2016

Related U.S. Application Data
(60) Provisional application No. 62/145,154, filed on Apr. 9, 2015.

(51) Int. Cl. A61F 2/16

(52) U.S. Cl. CPC A61F 2/1635; A61F 2/1618; A61F 2/1627; A61F 2002/1683

(57) ABSTRACT

In various embodiments, an intraocular lens features multiple compartments that, depending on the angular position of the lens, present different combinations of fluids to the central optical region of the lens to alter the refractive power of the lens.

### RELATED APPLICATION

This application claims the benefit of and priority to U.S. Provisional Patent Application No. 62/145,154, filed Apr. 9, 2015, the entire disclosure of which is hereby incorporated herein by reference.

### TECHNICAL FIELD

In various embodiments, the present invention relates to fluid-filled intraocular lenses with multiple filling fluids for adjustment of total refractive power.

### BACKGROUND

The crystalline lens of the human eye refracts and focuses light onto the retina. Normally the lens is clear, but it can become opaque (i.e., develop a cataract) due to aging, trauma, inflammation, metabolic or nutritional disorders, or exposure to radiation. While some lens opacities are small and require no treatment, others may be large enough to block significant fractions of light and obstruct vision.

Conventionally, cataract treatments involve surgically removing the opaque lens matrix from the lens capsule using, for example, phacoemulsification and/or a femtosecond laser through a small incision in the periphery of the patient's cornea. An artificial intraocular lens (IOL, or simply “lens”) may then be implanted in the lens capsule bag (the so-called “in-the-bag implantation”) to replace the crystalline lens (see, e.g., U.S. patent application Ser. No. 14/058,634, filed Oct. 21, 2013, the entire disclosure of which is incorporated by reference herein).

Generally, IOLs are made of a foldable, optically transparent polymeric material, such as silicone or acrylic, for minimizing the incision size and required stitches and, as a result, the patient's recovery time. The most commonly used IOLs are single-element lenses (or monofocal IOLs, non-accommodating IOLs, or non-focusing IOLs) that provide a single focal distance for distance vision. Typically, distance vision requires limited contraction of ciliary muscles in the eye (i.e., emmetropia); thus monofocal IOL designs are relatively simple. For example, to choose an appropriate geometry for a monofocal lens having a desired focusing power, limiting factors of the eye's anatomy, such as the axial eye length and the power of the cornea, are taken into consideration. However, because the focal distance is not adjustable following implantation of the monofocal IOL, patients implanted with such lenses can no longer focus on objects at a close distance (e.g., less than twenty-five centimeters); this results in poor visual acuity at close distances.

Most IOLs are made of single piece of hard material, although some newer IOLs have a two-lens design, and lenses filled with clear fluid have also been utilized. Most current IOLs are prefabricated for their lens power and then placed in the eye, but again, a few designs involve intraocular filling of the liquid in the lens at the time of initial surgery or possibly at a subsequent time (e.g., for adjustment or should the liquid become opacified, or even simply to exchange the liquid in the lens for a liquid of different properties (e.g., optical, viscosity, color)). A liquid-filled bag that provides accommodation—made from, for example, an elastic, biocompatible polymer—results in numerous benefits and advantages, e.g., the ability to adjust the lens following implantation; to customize the lens to the needs of each patient; to accommodate vision; sharper vision over a wide range of distances; and reduction of visual side effects such as glares and halos. See, e.g., U.S. Pat. No. 8,771,347, and U.S. patent application Ser. No. 13/473,012, filed May 16, 2012, the entire disclosure of each of which is hereby incorporated by reference.

Presbyopia-correcting lenses have been used to provide a larger range of viewing focus. Multifocal intraocular lenses simultaneously project both near and far focus distances on the retina, allowing the patient to have both a near in-focus image and a far in-focus image. However, due to the multiple focal planes, these lenses deleteriously exhibit visual disturbances such as halo or glare. Accommodating intraocular lenses adjust focus using the eye's natural focusing mechanism; such designs are promising, but no long-term solution with high levels of accommodation has been successfully implemented.

In view of the foregoing, there is a need for IOLs that provide variable focal lengths without the disadvantages of conventional lenses.

### SUMMARY

In accordance with embodiments of the present invention, IOLs contain two or more fluids within internal compartments defined by one or more septa. The septa thus separate the different fluids, thereby preventing mixing and resulting haziness of a patient's vision. Advantageously, fluids of similar polarity or surface energy may be utilized in close proximity within the IOL without degradation of optical quality that may result from fluid mixing. In various embodiments, the IOL can shift abruptly from one focal length to another, or the IOL can shift progressively through two or more focal lengths as the patient adjusts his or her eye.

In various embodiments of the invention, the septa within the IOL substantially prevent hazing that may result from mixing of the fluids within the IOL either at a meniscus that would otherwise form between the fluids or via more generalized mixing. In the absence of the septa, the meniscus that might form between fluids may be unstable and result in fluctuations or visible disturbances in the patient's vision. The septa separating the various chambers within IOLs in accordance with embodiments of the invention may be substantially impermeable or semipermeable to the fluids contained within the chambers.

In accordance with various embodiments of the invention, the fluids within the IOL may have different indices of refraction and/or densities. Thus, positional (e.g., angular) changes of the IOL may be utilized to preferentially drive one or more of the fluids to particular positions and/or chambers within the IOL and thereby alter the optical properties of various portions of the IOL. For example, fluids having different refractive indices may be moved into and out of the optical portion (e.g., optical axis) of the IOL to alter the overall IOL accommodative power (or simply “power”). In various embodiments, the movement of one or more of the fluids within the IOL alters the curvature of at least a portion of the IOL surface, thereby altering the lens power. In another exemplary embodiment, movement of one or more fluids within the IOL transforms the IOL from a monofocal lens when the patient is looking up or straight ahead to a multifocal lens when the patient is looking down. Such an arrangement provides the advantage of a monofocal lens without the negative visual disturbances of a multifocal lens at distance viewing, with the additional advantages of a multifocal lens when looking down, for example, when reading.

IOLs in accordance with embodiments of the invention may be substantially completely or only partially filled with fluid. In various embodiments, the IOL is primarily solid, and fluid channels are utilized to move fluid from one portion of the IOL to one or more other portions. For example, a substantially solid IOL may contain a fluid channel that leads to an internal optic. As the lens is positioned inferiorly (i.e., angled downward), one fluid may travel to the internal optic to alter refractive power. When the position (e.g., angle) of the lens changes, another fluid may fills the chamber, or the chamber may be closed. In addition, IOLs in accordance with embodiments of the invention may have valves (e.g., patch valves, duck-bill valves, multi-layer valves, etc.) connected to one or more of the fluid chambers, thereby allowing lenses to be injected into the eye and subsequently filled, refilled, accessed at a later date and titrated to correct fill, or fluid(s) exchanged and/or modified to adjust optical properties (e.g., by a needle or other filling device interfacing with the valve). Such valves may be self-sealing via various means, e.g., as described in U.S. patent application Ser. No. 14/980,116, filed on Dec. 28, 2015, the entire disclosure of which is incorporated by reference herein. Embodiments of the invention may utilize two or more internal chambers within an IOL and/or two or more different fluids within the IOL.

In an aspect, embodiments of the invention feature an intraocular lens having an optical axis and a central optical region disposed through the lens along the optical axis. The intraocular lens includes, consists essentially of, or consists of an outer membrane defining an interior region, a septum dividing the interior region into first and second fluidically separate chambers, a first fluid disposed within the first chamber, and a second fluid, different from the first fluid, disposed within the second chamber. The first fluid has a first density and a first refractive index. The second fluid has a second density and a second refractive index. The first and second densities may be substantially the same as each other or different from each other. The first and second refractive indices may be substantially the same as each other or different from each other. When the optical axis of the intraocular lens is approximately horizontal, light rays passing through the outer membrane along the optical axis pass through the first fluid without passing through the second fluid, whereby the intraocular lens has a first refractive power. When the optical axis of the intraocular lens is tilted downward, light rays passing through the outer membrane along the optical axis pass through the first fluid and through the second fluid, whereby the intraocular lens has a second refractive power different from the first refractive power.

Embodiments of the invention may include one or more of the following in any of a variety of combinations. The septum may be flexible. The second density may be larger than the first density. The first and second refractive indices may be different. The second refractive index may be larger than the first refractive index. The septum may be affixed to the outer membrane at (i) a first point at an anterior surface of the intraocular lens, the first point being disposed above the optical axis, and/or (ii) a second point at a posterior surface of the intraocular lens, the second point being disposed below the optical axis. The posterior surface of the outer membrane may have no optical power. The outer membrane may be flexible.

In another aspect, embodiments of the invention feature an intraocular lens having an optical axis and a central optical region disposed through the lens along the optical axis. The intraocular lens includes, consists essentially of, or consists of an outer membrane defining an interior region, a hollow secondary optic, a first fluid disposed within the interior region, at least one reservoir fluidically coupled to the secondary optic, and a second fluid different from the first fluid. At least one said reservoir may be disposed within the interior region. At least one said reservoir may be disposed outside of the outer membrane. The optical axis of the intraocular lens intersects the secondary optic. The second fluid is disposed within the at least one reservoir. The first fluid has a first density and a first refractive index. The second fluid has a second density and a second refractive index. The first and second densities may be substantially the same as each other or different from each other. The first and second refractive indices may be substantially the same as each other or different from each other. When the intraocular lens is tilted, second fluid is exchanged between the secondary optic and the at least one reservoir, thereby altering a refractive power of the intraocular lens.

Embodiments of the invention may include one or more of the following in any of a variety of combinations. At least one said reservoir may be collapsible. At least one said reservoir may be disposed outside of the central optical region of the intraocular lens. At least one said reservoir may be disposed within the central optical region of the intraocular lens. At least one conduit may fluidically couple the at least one reservoir and the secondary optic. The secondary optic may be disposed within the interior region. The secondary optic may be spaced away from the outer membrane. The secondary optic may be in direct contact with, partially defined by, or fully defined by the outer membrane. The secondary optic may be disposed on an external anterior surface of the intraocular lens. The secondary optic may be disposed on an internal anterior surface of the intraocular lens. The secondary optic may be disposed on an external posterior surface of the intraocular lens. The secondary optic may be disposed on an internal posterior surface of the intraocular lens. The second density may be larger than the first density. The first and second refractive indices may be different. The second refractive index may be larger than the first refractive index. The outer membrane may be flexible. The secondary optic may include, consist essentially of, or consist of a plurality of portions (e.g., concentric rings). The portions may be fluidically coupled to each other. The portions may be fluidically separate from each other (and may each have, e.g., separate filling valves). The at least one reservoir may include, consist essentially of, or consist of a plurality of reservoirs. Only one or more regions of the secondary optic may be configured to accept second fluid. The secondary optic may have a non-uniform shape relative to the optical axis. A surface of the secondary optic (e.g., a surface facing anteriorly and/or away from the outer membrane) may be substantially planar when the secondary optic is partially or substantially completely filled with second fluid.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean ±10%, and in some embodiments, ±5%. The term “consists essentially of ” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS. 1A-1E are schematic cross-sections of intraocular lenses with internal septa in accordance with embodiments of the invention;

FIGS. 2A and 2B are schematic cross-sections of an intraocular lens with an internal septum in accordance with embodiments of the invention;

FIGS. 3A and 3B are schematic cross-sections of an intraocular lens with an expandable anterior optic in accordance with embodiments of the invention;

FIG. 4 is a schematic cross-section of an intraocular lens with an internal optic and collapsible reservoir in accordance with embodiments of the invention;

FIGS. 5A and 5B are schematic cross-sections of an intraocular lens with an exterior expandable optic in accordance with embodiments of the invention;

FIGS. 6A and 6B are schematic cross-sections of an intraocular lens with an exterior expandable optic in accordance with embodiments of the invention;

FIG. 7 is a schematic plan view of a secondary optic for an intraocular lens in accordance with embodiments of the invention;

FIG. 8A is a schematic cross-section of an intraocular lens with an internal optical chamber in accordance with embodiments of the invention; and

FIG. 8B is a schematic plan view of the intraocular lens of FIG. 8A.

### DETAILED DESCRIPTION

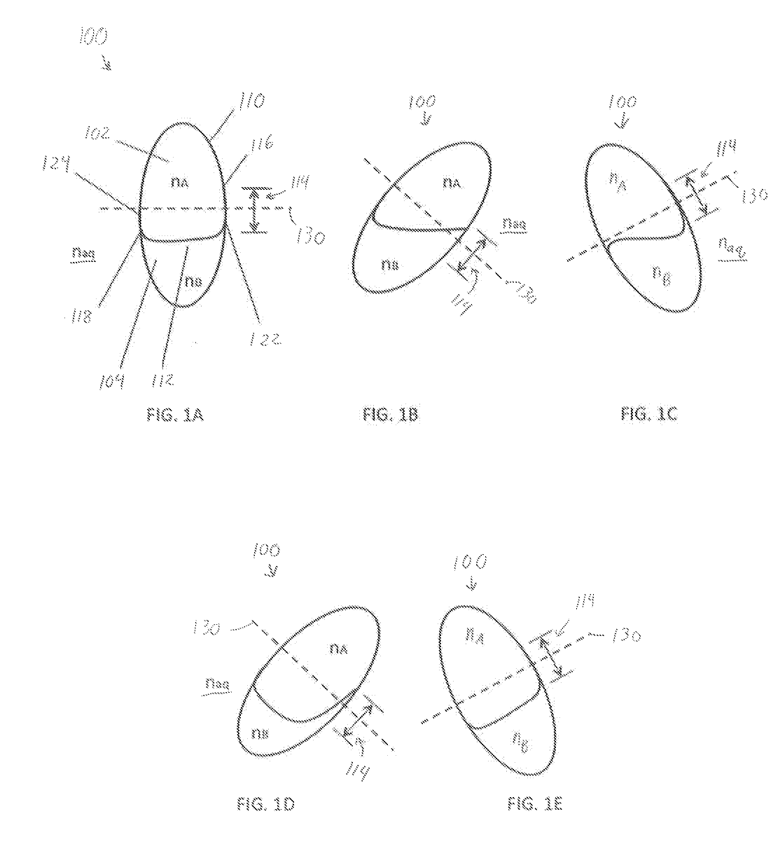

Refer to FIG. 1A, which depicts a liquid-filled intraocular lens 100 containing two filling fluids in two separate compartments. Liquid-filled intraocular lens 100 includes, consists essentially of, or consists of an outer wall 110 with a septum 112. Septum 112 separates a superior compartment 102 from an inferior compartment 104. Superior and inferior compartments 102, 104 are partially or substantially completely filled with fluid A and fluid B respectively. Fluid B has a higher density than fluid A, and fluid A and fluid B have differing refractive indices. In various operating conditions, optical rays pass through a central optical portion 114 of lens 100 that includes the optical axis 130 of the lens. As shown in FIG. 1A, as rays pass through central optical portion 114 they encounter the anterior surface 122 of lens 100, pass through compartment 102 and fluid A, and then through posterior surface 124. Therefore, the optical power of the lens is determined by the anterior and posterior radii of curvature along with the refractive index of fluid A. For example, anterior surface power may be defined as:

$$\frac{{n}_{A} - {n}_{aq}}{( {n}_{aq} ) ( {r}_{ant} )} ,$$

where n_{A} is the refractive index of fluid A, n_{aq} is the refractive index of the surrounding aqueous humor, and rₐₙₜ is the radius of curvature of the anterior surface 122 of the lens 100. Similarly, the refractive power of the posterior surface may be defined as:

$$\frac{{n}_{aq} - {n}_{A}}{( {n}_{aq} ) ( {r}_{post} )}$$

where rₚₒₛₜ is the posterior radius of curvature of the lens. Note that by convention, when the lens is biconvex, then rₚₒₛₜ is negative and rₐₙₜ is positive. Thus, if n_{A}>n_{aq}, both the anterior and posterior surface powers are positive.

FIG. 1B depicts the fluid-filled intraocular lens 100 as it is tilted. Fluid B tends to stay level with gravity and light passing through the lens 100 now passes through anterior surface 122, chamber 104 filled with fluid B, chamber 102 with fluid A and posterior surface 124. In this situation, anterior surface power may be defined as:

$$\frac{{n}_{B} - {n}_{aq}}{( {n}_{aq} ) ( {r}_{ant} )} ,$$

where n_{B} is the refractive index of fluid B. Posterior surface power remains the same as defined above:

$$\frac{{n}_{aq} - {n}_{A}}{( {n}_{aq} ) ( {r}_{post} )} .$$

If n_{B}>n_{A}, then when fluid B is on the anterior surface of the lens 100 through which light rays pass, the total surface power of the lens is higher than when only fluid A is in contact with the anterior surface. In this manner, the total lens power may be modulated based on the tilt of the lens 100. As utilized herein, the term “tilt” refers to an orientation with respect to the vector effect of gravity. In accordance with various embodiments described herein, the tilt may be affected only in one axis or in a combination of two or three axes of orientation. Other factors including specific gravity of the fluids used, momentum, inertial effects, etc. may also be taken into consideration in continuous fluid movement but may be adjusted by altering one or more of the characteristics of the fluid (e.g., viscosity).

In order for fluid A and fluid B to move appropriately, in various embodiments of the invention septum 112 is deformable. In certain configurations, as shown in FIGS. 1A-1E, the septum extends superiorly through the central optical portion 114 and is fixed against the wall of lens 100 at a point 116 on the anterior surface of lens 100 to allow fluid B to substantially completely cover central optical portion 114, at least along the wall, when lens 100 is tilted. Along the posterior surface of the lens 100, septum 112 attaches inferiorly to the wall at a point 118. (As utilized herein, the anterior surface of a lens faces outward from the patient's eye and the posterior surface faces inward toward the patient's retina; the optical axis passes through both surfaces.) As shown, in various embodiments point 118 is disposed below the central optical portion 114, thereby substantially preventing fluid B from interacting with the posterior surface of the lens 100, at least at the optical axis 130, when the lens 100 is tilted superiorly as shown in FIG. 1C. In various embodiments, point 116 is disposed above at least a portion of the central optical portion 114 and above the optical axis 130 (and thus above point 118 ).

FIG. 1D depicts lens 100 with a more rigid septum 112 tilted to look inferiorly, and FIG. 1E depicts the same lens tilted to look superiorly. (As used herein, the term “inferior” generally refers to a downward angle and the term “superior” generally refers to an upward angle.) The septum 112 in these two pictures is free to rock side to side between these two states. When looking inferiorly, fluid pushes the septum 112 to the posterior of the lens, and fluid B forms a lens with the anterior surface of lens 100. When the lens is tilted superiorly, the more rigid membrane rocks to the anterior portion of the lens. This design prevents fluid B from being disposed in the optical path when looking superiorly. Other thickness profiles as well as retaining features, such as struts or flexible spokes that attach the membrane to the walls of the lens, may be used to further restrain the motion of septum 112 if desired.

In various embodiments of the invention, the posterior side of the lens has no optical power (i.e., the surface is substantially flat so that the radius of curvature is infinite). Even though fluid B comes into contact with the flat posterior surface when looking superiorly, there is no refractive power change due to the infinite radius of curvature.

Central optical portion 114 is preferentially at least 2 mm in diameter, in certain configurations 4.25 mm in diameter, and in certain embodiments, at least 6 mm in diameter. In various embodiments, the optical axis approximately bisects the central optical portion 114 (and/or the intraocular lens itself) into approximately equal portions.

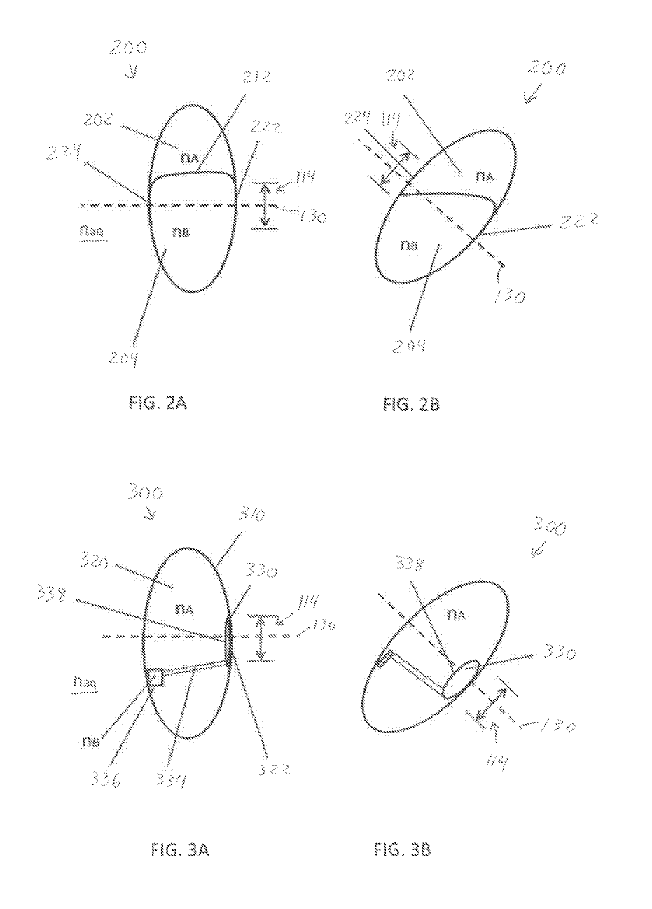

FIGS. 2A and 2B depict a fluid-filled intraocular lens 200 in accordance with embodiments of the present invention. Septum 212 separates fluid A and fluid B in chambers 202 and 204 respectively. When lens 200 is level, optical rays pass through central optical portion 114 and only pass through fluid B. When lens 200 is tilted, as shown in FIG. 2B, optical rays pass through anterior surface 222 and fluid B then through septum 212, fluid A, and posterior surface 224. As shown, the septum 212 is connected to anterior surface 222 at a point above optical axis 130 (and, in some embodiments, above central optical region 114), and the septum 212 is connected to the posterior surface 224 at a point below the optical axis 130 (and, in some embodiments, above central optical region 114). Overall refractive power of the lens changes in a similar manner as discussed with FIGS. 1A-1E. In various embodiments, fluid A may have a higher refractive index than fluid B to increase overall power.

In various embodiments of the invention, one or more portions of the septum may be biased to maintain a specific curvature (e.g., a curvature molded into its resting state). Along with a combination of anterior surface and posterior surface connection points, a variety of overall refractive power changes may be produced.

The fluid characteristics may be accessed during implantation, and numerous times post-operatively through independent self-sealing refill valves to alter fluid characteristics and therefore the refractive characteristics. Specific factors including fluid A and fluid B ratios, fill volume of each chamber, fill percentage, chamber pressure, balance of each chamber's pressure on the septum, and viscosity of each fluid may further affect the range of overall refractive power change of the lens between various tilts as well as the rate of change of refractive power due to tilt. Such fluid characteristics may have supplemental effects to accommodation for accommodative liquid-filled IOLs as disclosed herein.

Although in various embodiments the power of the lens increases when tilted, this need not be the case. For example, if the anterior lens surface is convex then rₐₙₜ is negative. If n_{B}>n_{A}, then when the lens is tilted and fluid B contacts the anterior surface, the anterior surface power becomes more negative, and overall lens power decreases. Likewise, if the anterior lens surface is convex and n_{A}>n_{B}, then as fluid B comes into contact with the anterior lens surface, total power decreases.

The intraocular lens may include one or more additional optical elements. FIGS. 3A and 3B depict a lens 300 with an expandable anterior optic 330 enclosed by an anterior surface 322 of the lens and a posterior septum 338. Outer wall 310 encloses a chamber 320. A collapsible reservoir 336 contains fluid B in fluidic continuity with expandable anterior optic 330 through a fluid line (or fluid conduit) 334. This allows fluid B to travel between anterior optic 300 and collapsible reservoir 336. Chamber 320 is otherwise partially or substantially filled with fluid A. As shown, in various embodiments, the collapsible reservoir 336 is disposed within the lens 300 but outside of the central optical region 114 and thus typically does not substantially affect the lens power regardless of the orientation of the lens. In contrast, optic 330 is at least partially disposed within the central optical region 114; all or a portion of optic 330 may be disposed within the central optical region 114, and all or a portion of central optical region 114 may be occupied, at least proximate the wall 310, by the optic 330. In various embodiments, fluid B is denser than fluid A. When lens 300 is level, collapsible reservoir 336 is inferior to expandable anterior optic 330 and fluid B remains in collapsible reservoir 336. When lens 300 is tilted forward, as depicted in FIG. 3B, collapsible reservoir 336 allows fluid B to flow through fluid line 334 to anterior optic 330, causing posterior septum 338 to expand. If fluid A and fluid B have differing refractive indices, the overall power of lens 300 changes. To illustrate this, if anterior optic A is convex, n_{B}>n_{A}, and n_{A}>n_{aq}, then lens power increases as the lens 300 is tilted down. As described above, other permutations based on refractive index of fluid A, fluid B, and the radii of curvature of the lens surfaces may result in the power increasing, decreasing, or remaining substantially constant.

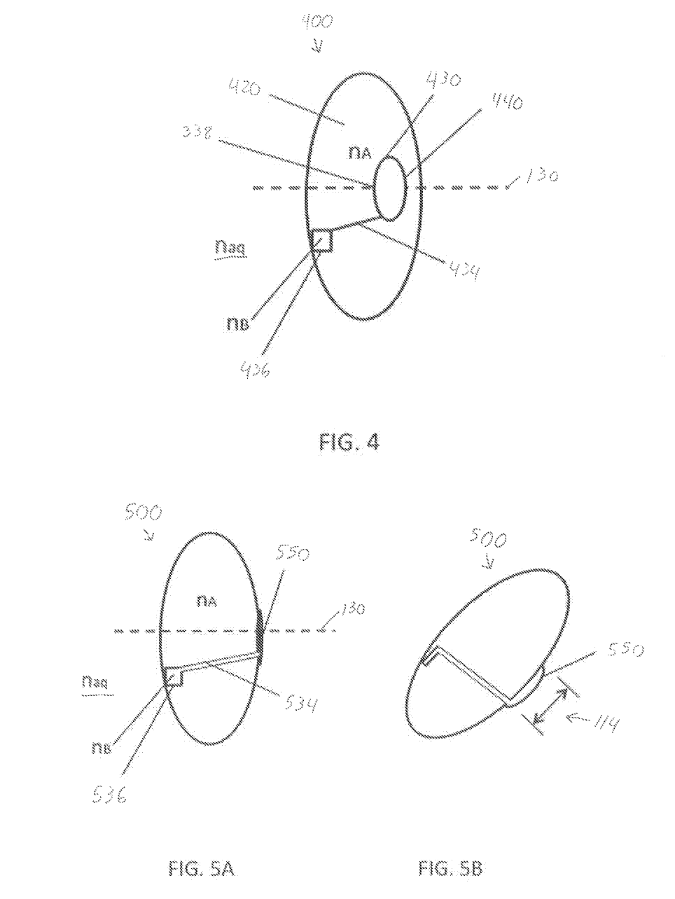

As shown in FIG. 4, the expandable optic may be internal to the intraocular lens 400. Here an internal lens (or “optic”) 430 is fluidly coupled to a collapsible reservoir 436 via a fluid line 434. Internal lens 430, collapsible reservoir 436, and fluid line 434 define a closed system filled with fluid B. Compartment 420 is otherwise substantially filled with fluid A. As lens 400 tilts, fluid B moves between collapsible reservoir 436 and internal lens 430. As detailed above, fluid B moves based on a difference in density between fluid B and fluid A. By filling internal lens 430, the refractive power of the overall lens 400 changes, assuming a difference in refractive index of fluid A and fluid B. As shown in FIG. 4, lens 430 is an enclosed chamber within lens 400 that may be spaced away from the wall enclosing chamber 420 and that may not be partially defined by the wall. The fluid line 434 may additionally integrate fluid flow restrictors and/or flow regulation valves to control the rate of change of the internal lens 430 with tilt, thereby creating desirable refractive index changes (e.g., smooth, and less noise due to naturally occurring flux of flow).

In still another alternative, the expandable optic may be external or integrated within the envelope membrane of the intraocular lens. Thus, FIGS. 5A and 5B depict an intraocular lens 500 with an additional separate expandable anterior surface (or “optic”). Lens 500 features a collapsible reservoir 536, a fluid line 534, and an anterior lens 550, any or all of which contain fluid B. Fluid A partially or substantially completely fills the other portions of the lens. In various embodiments of the invention, the anterior wall of lens 500 forms at least a portion of the anterior lens 550; that is, anterior lens 550 is enclosed by the anterior wall of lens 500 and an additional outer membrane that may include, consist essentially of, or consist of the same material as the wall of lens 500. In various embodiments, the anterior lens 550 is integrated within the outer wall of lens 500, i.e., portions of the outer wall surround a region fillable with fluid B. In various embodiments, anterior lens 550 is flush with the anterior lens surface of the lens 500 and is partially or substantially completely filled with fluid B when lens 500 is tilted, as shown in FIG. 5B. This expansion causes a change in the radius of curvature of the anterior portion of the lens 500 (at least in central optical region 114) as anterior lens 550 bulges outward, which in conjunction with the refractive properties of fluid A and fluid B causes the power of lens 500 to change. In other embodiments, a shape change occurs on the posterior portion of the lens 500 (i.e., lens 550 is disposed on or proximate the posterior wall of lens 500) and/or due to a shape change of another internal lens. In an alternative embodiment, the anterior lens 550 is formed flush on the interior surface of lens 500. As the lens 500 is tilted, the anterior lens 550 expands, altering the posterior surface of the anterior lens 550 and the associated radius of curvature. Thus, in accordance with various embodiments of the invention, the anterior lens 550 adds one or more radius of curvatures to the calculation of the anterior surface power. The anterior lens 550 may be formed flush with the lens 500 surface and with a predetermined diameter that may be further reinforced during expansion and contraction by a mechanical restraint (e.g. one or more rings). In such embodiments, the anterior lens 550 is normally filled by a known volume and the collapsible reservoir 536 is further expandable when the lens is tilted superiorly as in the orientation depicted in FIG. 1C. In various embodiments, the anterior lens 550 extends partially or substantially completely over the central optical region 114 of the lens 500.

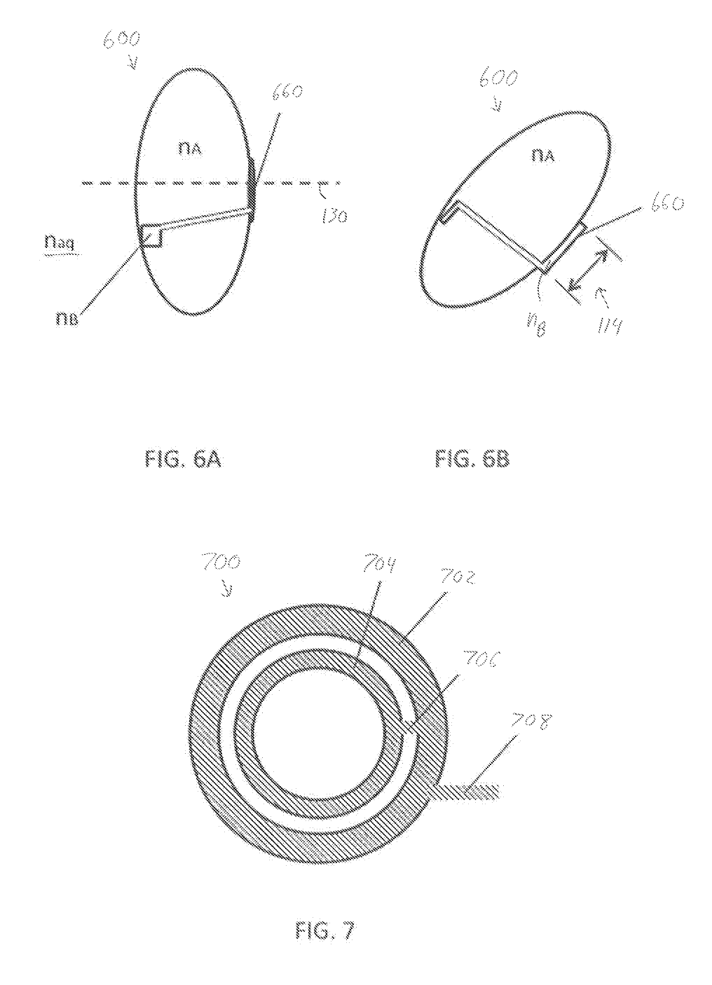

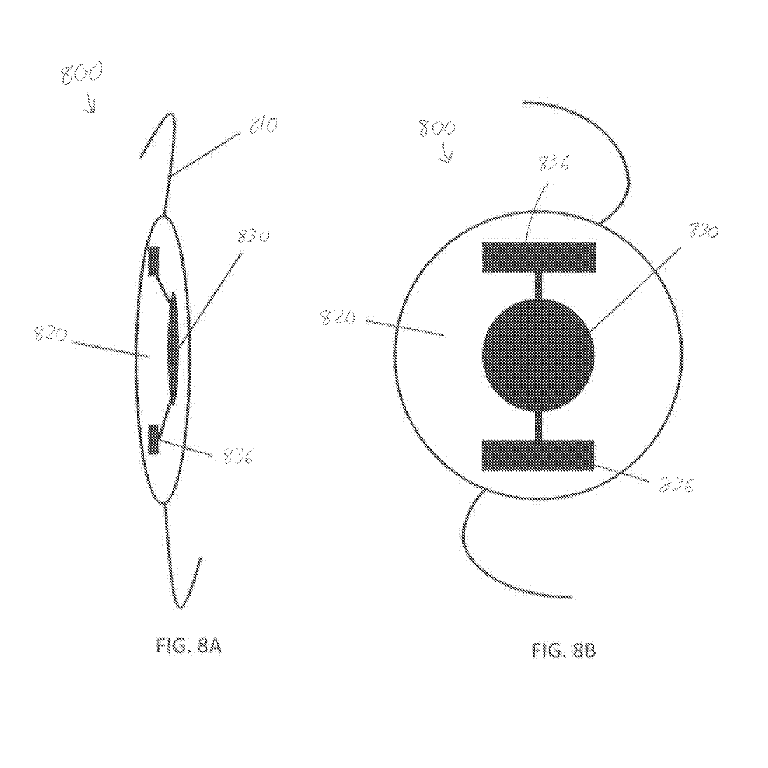

Filling of the additional optic may deform or otherwise change its curvature rather than or in addition to changing its volumetric shape. FIGS. 6A and 6B depict a lens 600 in accordance with embodiments of the invention and which has an anterior lens (or “optic”) or surface 660 with a different boundary condition from that of anterior lens 550 described above. Anterior lens 660 progresses from a more curved configuration to a less curved (e.g., substantially planar) configuration as it is filled, as shown in FIG. 6B. This may be achieved by altering the connection of the anterior lens 660 with the wall of the lens 600. To achieve specific deformation of the anterior lens 660, it may be connected with lens 600 with a point on the center of the optical axis 130, and/or by varying the thickness of lens 660 so that it expands anisotropically. Thickness variation or custom thickness profile of lens 660 may be used to correct for aberration or higher order aberration (e.g., counteracting astigmatism aberration in the cornea thereby acting as a toric lens or increasing spherical aberration to increase depth of field). In other embodiments, lens 660 increases aberration of the lens, for example, to increase overall depth of field of the lens. For example, the thickness in various regions may be varied (e.g., anisotropically), and, via variations of the fluid volume and/or the fluid pressure acting on such a variable-thickness surface, various portions may stretch or change differently.

The additional fillable optic need not present an uninterrupted surface. It may, for example, have an annular or other non-uniform shape relative to the optical axis. That is, optical rays passing through the additional optic need not all intersect a fillable region of thereof. Rather, the optic may have two or more discrete (and, in various embodiments, fluidly interconnected) regions that collectively consume only a portion of the surface area of the optic and thus only intercept (and, e.g., redirect and/or shape) some of the optical rays passing through the additional optic. Refer to FIG. 7, which depicts a complex secondary lens (or “optic”) that may be placed on the anterior or posterior portion of an intraocular lens in accordance with various embodiments of the present invention. The secondary lens 700 is in fluidic continuity with a fluid B in one or more of the manners detailed above (e.g., transferring fluid B to and from an expandable reservoir), while the intraocular lens on which lens 700 is disposed is otherwise partially or substantially completely filled with fluid A having a different density and/or refractive index. However, when filling fluid B enters the secondary lens 700, it does not necessarily cover the entirety of the surface of the intraocular lens on which lens 700 is disposed, or even the entirety of the surface area defined by the outer periphery of secondary lens 700. The secondary lens 700 may feature one or more rings or other shapes that accept fluid B but to not cover the entirety of the surface area of secondary lens 700. For example, FIG. 7 depicts a secondary lens 700 having an outer ring 702 and an inner ring 704 that are in fluidic connection via a fluid line (or conduit) 706. (As used herein, the term “ring” is used to connote a closed shape that is not necessarily circular; rather, a ring may be, e.g., elliptical or irregular in shape.) Fluid B may flow into and out of secondary lens 700 via a fluidic connection 708. As depicted in FIG. 7, when the secondary lens 700 is filled with fluid B, then outer ring 702 and inner ring 704 may be used to alter overall optical power of certain portions of the lens, in a manner similar to a multifocal lens. For example, the certain portions of the lens may include different optical correction zones tailored to improve specific types of vision (e.g. distance-dominant, intermediate, near, low light, bright light, etc.). The secondary lens 700 may increase or decrease the size of certain optical correction zones of the intraocular lens by altering the inner diameter and outer diameter of each ring by altering the fluid volume. Therefore, filling the secondary lens 700 with fluid B provides multifocality to the intraocular lens. If the intraocular lens is monofocal when the secondary lens 700 is not filled, the intraocular lens may alternate between monofocality and multifocality via filling of secondary lens 700, thus providing the advantages of a monofocal IOL when looking far and multifocality when looking near. This technique addresses many of the limitations of multifocal lenses such as visual disturbances when looking far or during night driving. Outer ring 702 and inner ring 704 may inflate to alter radius of curvature when filled with fluid B.

The expandable optic may be filled by, and drain into, more than one internal reservoir. FIGS. 8A and 8B depict a substantially solid intraocular lens 800 with two internal reservoirs. Internal optical chamber 830 is filled with fluid depending on the angle of the lens 800, as detailed herein. When the internal optical chamber 830 is filled, the overall refractive power of the lens 800 is altered. Two or more reservoirs 836 are used to store fluid(s) to fill the internal optical chamber 830. In various embodiments, lens 800 may be shaped similar to a conventional intraocular lens, and may have one or more haptics 810. The haptics 810 hold the lens 800 within the capsular bag inside the eye. The anterior and/or posterior surfaces of lens 800 may have refractive powers and may correct for optical aberration, or provide multifocality (e.g. diffractive or refractive, possibly apodized), as detailed herein. In various embodiments, the volume, shape, and/or curvature of the lens 800 and/or the chamber 830 do not change as a function of the amount of fluid fill within chamber 830. Thus, the volume and/or type of filling fluid may be utilized to alter the refractive power of 800 as a function of orientation (e.g., tilt) of the lens 800.

Although the lens orientations have been described herein primarily as looking inferiorly or approximately horizontally, this is not meant to limit the scope of the present invention. Many more configurations are possible using similar techniques without undue experimentation by those skilled in the art. A few non-limiting examples include switching refractive power at a position other than horizontal, e.g. when looking superiorly or an intermediate state. Alternatively, when level, lenses in accordance with embodiments of the invention may be in an intermediate state between near vision and far vision. Intermediate states of a lens may provide a multifocality of the lens. In such embodiments, the lens is viewed as an alternating multifocal lens. As an example, as one lens fills with a fluid, the overall corrective power of the lens switches from primarily far vision to primarily near vision. However, as this process occurs, select portions of the lens may convey near vision, while other portions of the lens may transmit a far focal length.

Lenses in accordance with embodiments of the invention may be implanted with minimal or no volume within all chambers to decrease lens size and thus the incision size required to implant the lens within a patient's eye. The lens chambers and collapsible reservoirs may each contain one or more valves accessible from an external portion of the lens with a needle or other fluid line for filling. Such valves may be self-sealing, e.g., as described in U.S. patent application Ser. No. 14/980,116, filed on Dec. 28, 2015, the entire disclosure of which is incorporated by reference herein.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An intraocular lens having an optical axis and a central optical region disposed through the lens along the optical axis, the intraocular lens comprising:
an outer membrane defining an interior region;
disposed within the interior region, a septum dividing the interior region into first and second fluidically separate chambers;
a first fluid disposed within the first chamber, the first fluid having a first density and a first refractive index; and
a second fluid, different from the first fluid, disposed within the second chamber, the second fluid having a second density and a second refractive index,
wherein (i) when the optical axis of the intraocular lens is approximately horizontal, light rays passing through the outer membrane along the optical axis pass through the first fluid without passing through the second fluid, whereby the intraocular lens has a first refractive power, and (ii) when the optical axis of the intraocular lens is tilted downward, light rays passing through the outer membrane along the optical axis pass through the first fluid and through the second fluid, whereby the intraocular lens has a second refractive power different from the first refractive power.

2. The lens of claim 1, wherein the septum is flexible.

3. The lens of claim 1, wherein the second density is larger than the first density.

4. The lens of claim 1, wherein the first and second refractive indices are different.

5. The lens of claim 1, wherein the second refractive index is larger than the first refractive index.

6. The lens of claim 1, wherein the septum is affixed to the outer membrane at (i) a first point at an anterior surface of the intraocular lens, the first point being disposed above the optical axis, and (ii) a second point at a posterior surface of the intraocular lens, the second point being disposed below the optical axis.

7. The lens of claim 1, wherein a posterior surface of the outer membrane has no optical power.

8. The lens of claim 1, wherein the outer membrane is flexible.

9. An intraocular lens having an optical axis and a central optical region disposed through the lens along the optical axis, the intraocular lens comprising:
an outer membrane defining an interior region;
a hollow secondary optic, the optical axis of the intraocular lens intersecting the secondary optic;
a first fluid disposed within the interior region, the first fluid having a first density and a first refractive index;
at least one reservoir disposed within the interior region and fluidically coupled to the secondary optic; and
disposed within the at least one reservoir, a second fluid, different from the first fluid, the second fluid having a second density and a second refractive index;
wherein, when the intraocular lens is tilted, second fluid is exchanged between the secondary optic and the at least one reservoir, thereby altering a refractive power of the intraocular lens.

10. The lens of claim 9, wherein the at least one reservoir is collapsible.

11. The lens of claim 9, wherein the at least one reservoir is disposed outside of the central optical region of the intraocular lens.

12. The lens of claim 9, further comprising at least one conduit fluidically coupling the at least one reservoir and the secondary optic.

13. The lens of claim 9, wherein the secondary optic is disposed within the interior region.

14. The lens of claim 13, wherein the secondary optic is spaced away from the outer membrane.

15. The lens of claim 9, wherein the secondary optic is in direct contact with, partially defined by, or fully defined by the outer membrane.

16. The lens of claim 9, wherein the secondary optic is disposed on an external anterior surface of the intraocular lens.

17. The lens of claim 9, wherein the secondary optic is disposed on an internal anterior surface of the intraocular lens.

18. The lens of claim 9, wherein the secondary optic is disposed on an external posterior surface of the intraocular lens.

19. The lens of claim 9, wherein the secondary optic is disposed on an internal posterior surface of the intraocular lens.

20. The lens of claim 9, wherein the second density is larger than the first density.

21. The lens of claim 9, wherein the first and second refractive indices are different.

22. The lens of claim 9, wherein the second refractive index is larger than the first refractive index.

23. The lens of claim 9, wherein the outer membrane is flexible.

24. The lens of claim 9, wherein the secondary optic comprises a plurality of concentric rings, the rings being fluidically coupled to each other.

25. The lens of claim 9, wherein the at least one reservoir comprises a plurality of reservoirs.

26. The lens of claim 9, wherein only one or more regions of the secondary optic are configured to accept second fluid.

27. The lens of claim 9, wherein the secondary optic has a non-uniform shape relative to the optical axis.

28. The lens of claim 9, wherein a surface of the secondary optic is substantially planar when the secondary optic is partially or substantially completely filled with second fluid.
